(19) United States
(12) Patent Application Publication
(10) Pub. No.: US 2011/0312947 A1
(43) Pub. Date: Dec. 22, 2011

(54) Method of improving ß-adrenergic receptor function

(75) Inventors: Richard S. Surwit, Durham, NC (US); Sheila A. Collins, Durham, NC (US)

(73) Assignee: Duke University, Durham, NC (US)

(21) Appl. No.: 13/064,238

(22) Filed: Mar. 11, 2011

Related U.S. Application Data
(63) Continuation of application No. 11/584,005, filed on Oct. 20, 2006, now abandoned.
Application No. 11/584,005 is a continuation of application No. 11/074,011, filed on Mar. 8, 2005, now abandoned.
Application No. 11/074,011 is a continuation of application No. 10/394,666, filed on Mar. 24, 2003, now abandoned.
Application No. 10/394,666 is a continuation of application No. 09/664,547, filed on Sep. 18, 2000, now abandoned.
(60) Provisional application No. 60/154,329, filed on Sep. 17, 1999.

(51) Int. Cl. A61K 31/5415; A61P 3/10; A61P 3/04

(52) U.S. Cl. 514/223.5

(57) ABSTRACT

The present invention relates to a method of increasing the sensitivity of a hyperinsulinemic patient to the anti-obesity and antidiabetic effects of a β adrenergic receptor (βAR) agonist. The method comprises administering to the patient the βAR agonist and an agent that reduces plasma insulin levels and thereby increases the sensitivity of the patient to the effects of the βAR agonist.

This application claims priority from Provisional Application No. 60/154,329, filed Sep. 17, 1999, the entire contents of that application being incorporated herein by reference.

### TECHNICAL FIELD

The present invention relates to a method of increasing the sensitivity of a hyperinsulinemic patient to the anti-obesity and antidiabetic effects of a β adrenergic receptor (βAR) agonist. The method comprises administering to the patient the βAR agonist and an agent that reduces plasma insulin levels and thereby increases the sensitivity of the patient to the effects of the βAR agonist.

### BACKGROUND

It has been repeatedly demonstrated that various in-bred strains of mice are differentially susceptible to developing obesity on high fat (HF) diets (Surwit et al, Diabetes 9:1163-1167 (1988), West et al, Am J. Physiol. 262:R1025-R1032 (1992). One strain that is particularly vulnerable to this type of diet-induced obesity is the C57BL/6J (B6) mouse. When placed on a HF diet. B6 mice develop severe obesity, insulin resistance, and hyperglycemia (Surwit et al, Diabetes 9:1163-1167 (1988). Surwit et al, Diabetes 40:82-87 (1991). Surwit et al, Metabolism 44:645-651 (1995)). Furthermore, B6 mice develop diet-induced obesity without increased caloric consumption or a reduction in their physical activity (Brownlow et al, Physiol. Behav. 60:37-41 (1996)). While the mechanism by which dietary fat induces diabetes and obesity in these mice is not understood, their obesity is characterized by adipocyte hyperplasia, particularly in the mesenteric fat pad and, at a molecular level, is accompanied by a loss of β₁- and β₃-adrenergic receptor (AR) expression and function in adipose tissue (Collins et al, Endocrinology 138:405-413 (1997)). Interestingly, this diet-induced impairment in the βARs is quite similar to what is observed in monogenic models of obesity such as Lep^{ob} (C57BL/6J-Lep^{ob}), LepR^{db} (129P3/J-LepR^{db-3J}), B6^{tub} (c57BU6J-tub) and Cpe^{fat} (C57BL/6J Cpe^{fat}/J) (Collins et al, Mol. Endocrinol. 8:518-527 (1994). Collins et al, Intl. J. Obes. 23:669-677 (1999)). In addition, it has been shown that B6 mice, when raised on a HF diet, appear to be relatively refractory to the effects of a selective β₃AR agonist (Collins et al, Endocrinology 138:405-413 (1997)). Since all three βAR subtypes stimulate lipolysis (Galitzky et al, Am. J. Physiol. 264:E403-E412 (1993). Galitzky et al, Fundam. Clin. Pharmacol. 9:324-331 (1995)) and the induction of the uncoupling protein 1 (UCP1) gene in brown adipose tissue (BAT) (Rohlfs et al. J. Biol. Chem. 270:10723-10732 (1995)) in response to catecholamines, we have hypothesized that defects in the β₃AR, the most abundant AR in rodent adipose tissue, are responsible for the development of diet-induced obesity and diabetes in B6 mice.

The molecular basis for this diet-induced change in βAR expression is not known. However, evidence is accumulating that hyperinsulinemia may play a role. Although genetic and dietary models of obesity display various endocrine abnormalities (Coleman, Diabetes 31:1-6 (1982). Coleman and Eicher, Journal of Heredity 81:424-427 (1990)), hyperinsulinemia is the one common feature among all of these models. In support of this idea, when differentiated 3T3-F442A mouse adipocytes were treated with insulin, β₃AR expression rapidly declined Fève et al, Proc. Natl. Acad. Sci. 91:5677-5681 (1994)). In addition, a role for insulin in affecting βAR function in adipocytes is supported by a series of studies showing that suppressing hyperinsulinemia with the K_{ATP} channel agonist, diazoxide (Dz), results in improved ability to stimulate lipolysis and a significant loss of adipose tissue mass (Alemzadeh et al. Metabolism 45:334-341 (1996), Alemzadeh et al, J. Clin. Endocrinol. Metab. 83:1911-1915 (1998)).

The present invention results from studies designed to test the hypothesis that the development of the obesity and diabetes syndrome in B6 mice raised on a HF diet is related to the hyperinsulinemia that arises in response to fat feeding. Applicants hypothesized that suppressing the development of hyperinsulinemia in B6 mice with Dz would result in both an improvement in the diabetes/obesity phenotype and a reversal in the loss of β₃AR expression and function in adipocytes. The invention further results from studies designed to determine whether suppression of hyperinsulinemia would enable a selective β₃AR agonist to ameliorate this diet-induced syndrome.

### SUMMARY OF THE INVENTION

The present invention relates to a method of increasing the sensitivity of a hyperinsulinemic patient to the anti-obesity and antidiabetic effects of a β adrenergic receptor (βAR) agonist. The method comprises administering to the patient the βAR agonist and an agent that reduces plasma insulin levels and thereby increases the sensitivity of the patient to the effects of the βAR agonist.

Objects and advantages of the present invention % will be clear from the description that follows.

### BRIEF DESCRIPTION OF THE DRAWINGS

FIG. 1. The effect of low fat (♦), high fat (▪), and high fat diets containing CL (□), Dz (◯), or CLDz () n the body weight of B6 mice. Mice were fed the high fat diet for 4 weeks before starting the treatments. Treatments with different superscripts are significantly different. a vs. b. p<0.001; a vs. c, p<0.001; b vs. c, p<0.01. Mice fed the low fat diet serve as a reference group of lean controls.

FIG. 2. The feed efficiency (body weight gain/kcal consumed) of B6 mice after consuming low fat (LF), high fat (HF), high fat+0.001% CL316,243 (CL) (disodium (R,R)-5-[2[[2-(3-chlorophenyl)-2-hydroxyethyl]-amino]propyl]-1,3-benzodioxole-2,2-dicarboxylate), high fat+0.32% diazoxide (Dz) or high fat+both compounds (CLDz) for 1 month. Treatments with different superscripts are significantly different. a vs. b. p<0.001: a vs. c, p<0.001: b vs. c, p<0.05. Mice fed the low fat diet serve as a reference group of lean controls.

FIG. 3. An estimate of the percent body fat in B6 mice fed low (LF). high fat (HF), high fat+0.001% CL316,243 (CL), high fat+0.32% diazoxide (Dz), or high fat+both compounds (CLDz) for 1 month. Percent body fat was estimated by the weight of epididymal fat as a proportion of the total body weight. See references (Collins et al, Obes. Res. 5:6S (1997), Rogers et al, Brit. J. Nutr. 43:83-86 (1980)) for validation of estimate. Treatments with different superscripts are significantly different. a vs. b, p<0.001: a vs. c, p<0.001; b vs. c, p<0.05. Mice fed the low fat diet serve as a reference group of lean controls.

FIGS. 4A and 4B. The effect of treatment on fasting plasma insulin (FIG. 4A) and glucose (FIG. 4B). Mice were fed a high fat diet for 1 month before the treatment began. Blood was collected before the treatment period started and biweekly thereafter. (♦) low fat, (▪) high fat, (□) high fat+0.001% CL316,243, (◯) high fat+0.32% diazoxide, () high fat+both compounds. Treatments with different superscripts are significantly different at day 31. (A) Insulin: a vs. b, p<0.001; a vs. c, p<0.001; b vs. c, p<0.05. (B) Glucose: a vs. b, p<0.001; a vs. c, p<0.001; b vs. c, p<0.001. Low-fat fed mice serve as a reference group of lean controls.

FIG. 5. The effect of treatment on glucose tolerance. Mice were injected with 0.5 g/kg of glucose. Blood samples were collected 30 minutes later and analyzed for glucose. (LF) low fat. (HF) high fat, (CL) high fat+0.001% CL316,243, (Dz) high fat+0.32% diazoxide, or (CLDz) high fat+both compounds. Treatments with different superscripts are significantly different. a vs. b, c, or d, p<0.001; b vs. c, p<0.01; b vs. d, p<0.001; c vs. d, p<0.05. Mice fed low fat serve as a reference group of lean controls.

FIG. 6. The effect of treatment on glucose transport into epididymal white and retroperitoneal adipose tissue as measured by the accumulation of ¹⁴C 2-deoxyglucose in the tissues. (LF) low fat, (HF) high fat, (CL) high fat+0.001% CL316,243, (Dz) high fat+0.32% diazoxide, or (CLDz) high fat+both compounds. Treatments with different superscripts are significantly different. a vs. b, p<0.05. The ANOVA for retroperitoneal fat was not statistically significant (p=0.07). Mice fed low fat serve as a reference group of lean controls.

FIG. 7. The stimulation of adenylyl cyclase activity by the β₃AR-selective agonist CL316,243 in membranes from animals fed the (♦), LF; (▪), HF; or (◯), Dz diets. The assays were incubated for 10 min. The cAMP produced was measured by radioimmunoassay. The data are expressed as picomoles of cAMP produced per mg of membrane protein per min of incubation. Curves represent the mean of three experiments for each condition. Non-linear regression analysis revealed that all three curves were significantly different from each other (p<0.0003).

FIG. 8. The effect of treatment on β₃AR mRNA levels in EWAT (epididymal WAT (white adipose tissue)). Forty μg of total cellular RNA from EWAT were fractionated through 1.2% agarose gels and blotted. The blot was probed with α-³²P-labeled β₃AR and with cyclophilin. Amounts of β₃AR mRNA were determined with a Molecular Dynamics phosphorimager and were normalized to cyclophilin mRNA levels. Treatments with different superscripts are significantly different. a vs. b, p<0.05. Mice fed low fat serve as a reference group of lean controls.

FIG. 9. Effect of treatment on UCP1 (uncoupling protein-1) levels in IBAT (interscapular BAT) and RP (retroperitoneal). Methods are as described in Collins et al, Intl. J. Obes. 23:669-677 (1999), except that blots were probed with α³²P-labeled UCP1 and cyclophilin. Treatments with different superscripts are significantly different. IBAT: a vs. b. p<0.05; a vs. c, p<0.001; b vs. c, p<0.01. RP: a vs. b, p<0.01.

### DETAILED DESCRIPTION OF THE INVENTION

The studies described in the Example that follows demonstrate that the K_{ATP} channel agonist diazoxide (Dz) greatly attenuates the development of obesity in B6 (C57BL/6J) mice raised on a high fat (HF) diet. Furthermore, Dz therapy completely reversed the development of diet-induced diabetes in these animals. While it has been previously shown that Dz treatment can induce weight loss in obese Zucker rats (Alemzadeh et al, Metab. 45:334 (1996)) and in humans (Alemzadeh et al, J. Clin. Endocrin. Metab. 83:1911 (1998)), the antidiabetic effect of Dz has not been demonstrated in a non-mutant model of diabetes. As in previous studies, Dz therapy reduced the fasting insulin levels without causing an increase in blood glucose. However in the present study, fasting glucose levels in treated animals were actually lower than those of control animals raised on a low fat (LF) diet. The body weights of the animals receiving either Dz alone or the combination therapy were significantly lower than the HF control animals. As in previous studies (Alemzadeh et al. Metab. 45:334 (1996)) there was an even greater effect on adipose tissue weights and overall percent body fat in animals treated with Dz. In the present study, there was a significant decrease in feed efficiency in Dz treated animals. Although it has been postulated that Dz affects appetite (Alemzadeh et al, Endocrin. 140:3197 (1999)), it was observed that the Dz-treated animals eat more, but gain less weight.

The mechanism by which Dz acts to reduce obesity and improve glucose metabolism is currently not known. Because Dz is a non-selective kATP channel agonist and may have other direct effects on metabolism, the observed effects cannot be conclusively attributed solely to a reduction in hyperinsulinemia. For instance, Alemzadeh et al (J. Clin. Endocrino. Metab. 83:1911 (1998)) have noted that Dz could have direct extrapancreatic effects on Ca+2 influx resulting in decreased lipogenesis. However, other data suggest that the reduction in circulating plasma insulin levels could directly improve β₃AR function in adipocytes (Fève et al, Proc. Natl. Acad. Sci. USA 91:5677 (1994)). For example, it has been previously shown that B6 mice, as well as other hyperinsulinemic models of obesity, exhibit decreased β₃AR expression and function (Collins et al, Intl. J. Obes. 23:669-677 (1999), Collins et al. Obes. Res. 5:6S (1997)). Furthermore, the B6 mice raised on a HF diet are comparatively insensitive to treatment with the selective β₃AR agonist CL (Collins et al, Obes. Res. 5:6S (1997)). In the present study, treatment with Dz resulted in an increase in the expression of the β₃AR and an improvement in the function, as evidenced by increased cAMP production in response to selective β₃AR-agonist stimulation. Also, animals treated with Dz showed an increase in expression of UCP1 in both IBAT and RP fat pads. This result was unexpected since no overt changes in β₃AR function were found in these tissues. at least as defined by adenylyl cyclase activation.

Interestingly, the combined therapy significantly increased glucose transport in EWAT, but not in muscle, suggesting that changes in insulin sensitivity in fat rather than muscle may be critical to the development of diabetes in the B6 mouse model. This is in agreement with previous work in which Dz has been shown to increase glucose transport in fat (Alemzadeh et al, Metab. 45:334 (1996)) and in studies in which transgenic mice that over-express GLUT4 selectively in adipose tissue exhibit a marked attenuation in streptozotocin-induced diabetes (Tozzo et al, Endocrin. 138:1604 (1997)). It has always been assumed that obesity somehow leads to insulin resistance, which in turn promotes hyperinsulinemia through excessive demand on the pancreatic beta cell. The present findings suggest a departure from this theory in which diet-induced obesity promotes hvperinslinemia which, in turn, leads to insulin resistance, possibly through diminished βAR function in adipose tissue. While the mechanism by which a HF diet initiates hyperinsulinemia is not known, it has been previously shown that HF feeding attenuates the insulin response to glucose in isolated islets from B6 mice, while the insulin response to lipid is less affected (Lee et al, Pancreas 11:206 (1995)). Further research is needed to determine if this defect in islet function is correctable with Dz.

In previous research, B6 mice raised on a HF diet proved relatively resistant to the anti-obesity effects of the selective β₃AR agonist CL (Collins et al. Endocrinology 138:405-413 (1997)). In the present study, diet-induced obese B6 mice treated with Dz actually show an enhanced response to CL316,243 (CL), indicating that the lack of efficacy of this compound previously observed in obese B6 mice is directly linked to the persistent hyperinsulinemia. The combined therapy was more effective in reducing feed efficiency, decreasing percent body fat, reducing circulating free fatty acids and improving glucose tolerance than either Dz or CL alone. While it is not immediately apparent as to why the combination was so much more effective than either therapy alone, the combined therapy did significantly increase glucose transport in epididymal (EWAT) and RP, as well as UCP1 expression in IBAT and RP fat depots when compared to the other treatments. The data presented here indicate that a combination of an insulin suppressing agent such as Dz with a β₃AR agonist provides a more potent therapy for diabetes and obesity than either treatment used alone.

Certain aspects of the present invention are described in greater detail in the non-limiting Example that follows.

### Example

### Experimental Details

Animals and Diets. Seventy-five 4-week old B6 male mice were obtained from Jackson Laboratories (Bar Harbor, Me.). The animals were housed 5 per cage in a temperature controlled (22° C.) room with a 12 hour light/dark cycle (lights off at 19:00). The HF and low fat (LF) experimental diets were manufactured by Research Diets (New Brunswick, N.J.), and contain 58% and 11% of calories from fat, respectively. The composition of these diets have been described in detail elsewhere (Surwit et al. Metabolism 44:645-651 (1995)). A group of 60 mice were fed the HF diet for the first four weeks of the study; the remaining 15 mice were fed the LF diet. The mice assigned to the LF diet were maintained on this diet throughout the study as a reference group of lean control mice. At week 4, all HF-fed mice were assigned to 4 groups of 15 mice each. The first group remained on the HF diet throughout the study as the “obese” control group. The remaining 3 groups of mice were fed the HF diet containing either the βAR agonist CL316,243 (CL), diazoxide (Dz) or the combination of the two compounds (CLDz). The CL concentration in the diets was 1 mg/kg (0.001%). This dose had originally been recommended by Dr. Thomas Claus, of Lederle Laboratories and found to be differentially effective in reducing obesity in obesity-prone B6 and obesity-resistant A/J mice (Collins et al, Obes. Res. 43:83-86 5:6S (1997)). The Dz concentration in the diets was 3.2 g/kg (0.32%) was determined to be the maximally effective dose for weight-reduction in dose-response pilot studies.

Body Weight, Food Intake, and Feed Efficiency. Animals were weighed weekly and food consumption measured per cage twice weekly until the diets were changed at week 4, whereupon body weight and food intake were determined daily excepting weekends. The feed efficiency (grams body weight gained/kilocalories consumed) was calculated on a per cage basis.

Glucose, Insulin, and Leptin. Samples for analysis of insulin, glucose, and leptin were collected as previously reported (Surwit et al, Metabolism 44:645-651 (1995), Collins et al, Endocrinology 138:405-413 (1997)) at day 24 (4 days before the diets were changed). at day 32 (4 days after the change) and biweekly thereafter. In all cases, food was removed 8 hours before samples were collected. Glucose was analyzed by the glucose oxidase method (Beckman Glucose Analyzer II). Insulin and leptin concentrations were determined by double antibody radioimmunoassay (Linco Research, St. Louis, Mo.). The insulin assay was based on a rat standard and the leptin assay used a mouse standard.

Triglycerides and Non-Esterified Fatty Acids. At the termination of the study, a postprandial plasma sample was collected and analyzed for triglyceride and non-esterified fatty acid (NEFA) concentrations using kits from WAKO Diagnostics (Richmond, Va.).

Tissue Collection. After 4 weeks of drug treatment. a subset of 10 animals from each group was killed. The epididymal white adipose tissue (EWAT). retroperitoneal (RP) fat, interscapular brown adipose tissue (MAT) fat pads and gastrocnemius muscle were removed, trimmed, weighed. and all tissues were flash-frozen in liquid nitrogen and stored at −80° C. for later determination of β₃AR, (EWAT) UCP1 expression (IBAT and RP), UCP2 and 3 expression (IBAT) as well as β₃AR-stimulated adenylyl cyclase activity (EWAT).

Percent Body Fat. The percent body fat was estimated from the weight of the epididymal fat pad. It has been shown that, as a proportion of total body weight, the epididymal fat pad weight is highly correlated with percent body fat (Rogers et al, Brit. J. Nutr. 43:83-86 (1980), Eisen et al. Growth 45:100-107 (1981)).

Glucose Tolerance Test. A subset of 5 animals from each group was injected intraperitoneally with 0.5 g/kg of glucose. At 30 minutes post injection, a plasma sample was collected and analyzed for glucose content by the glucose oxidase method.

Glucose Transport. Basal glucose transport into white adipose tissue and muscle was assessed following the method of Skillman and Fletcher (Skillman et al, Exp. Clin. Endocrinol. 87:288-298 (1986)) with minor modifications. Five mice from each group were injected i.p. with 50 mg/kg 2-deoxyglucose (20 μCi/kg ¹⁴C 2-deoxyglucose) mixed with 2-deoxyglucose (final specific activity was 66 μCi/mmole). Twenty minutes later the mice were killed by cervical dislocation. The thoracic cavity was opened and 5 mls saline was perfused through the left ventricle with the right atrium cut to diminish the vascular ¹⁴C levels. The right epididymal and RP fat pads and gastrocnemius muscle were excised, rinsed, blotted and weighed. The tissues were solubilized in 1 M NaOH at 37° C. overnight. The samples were neutralized with 1 M HCl and the ¹⁴C content determined.

Isolation and Analysis of RNA. Total cellular RNA was prepared using the tissues of 6 mice per group by the cesium chloride gradient method as detailed (Chirgwin et al. Biochemistry 18:5294-5299 (1979)). For Northern blot hybridization, RNA was denatured by the glyoxal procedure, fractionated through 1.2% agarose gels, and blotted onto Biotrans (ICN) nylon membranes (Collins et al, J. Biol. Chem. 263:9067-9070 (1988)). Radiolabeled probes were prepared by random primer synthesis (PrimeIT, Stratagene) of the purified DNA fragments in the presence of α[³²P]deoxy CTP to a specific activity>2×10⁹ dpm/μg DNA. A fragment specific for the mouse β₃AR was prepared as previously described (Collins et al. Mol. Endocrinol. 8:518-527 (1994)). For mitochondrial UCP1, a 300-bp Bgl I fragment, kindly provided by Dr. Leslie P. Kozak, was used (Kozak et al, J. Biol. Chem. 263:12274-12277 (1988)). A rat cDNA probe for cyclophilin was used as an internal hybridization/quantitation standard. Blots were hybridized and washed as previously described (Collins et al, Mol. Endocrinol. 8:518-527 (1994), (Thomas, Proc. Natl. Acad. Sci. USA 77:5201-5205 (1980)). The intensity of hybridization signals was quantified by PhosphorImager (ImageQuant/Storm) and normalized to the values for cyclophilin.

Preparation of plasma membranes and adenylyl cyclase assay. Adipose tissue was isolated from each group of animals. The tissues from 4 mice per group were pooled and minced and then plasma membranes prepared as previously described (Collins et al, Mol. Endocrinol. 8:518-527 (1994)). Adenylyl cyclase activity was measured using established methods, and the cAMP formed was measured by RIA (Harper et al, J. Cyclic Nucleotide Res. 1:207-218 (1975)) using a polyclonal anti-serum to cAMP (Gettys et al, J. Biol. Chem. 2.66:15949-15955 (1991)). Protein concentrations were determined by the Bradford method (Bradford. Anal. Biochem 72:248-254 (1976)).

Statistical Analyses. Unless otherwise noted, data was analyzed using ANOVA. All p values less than 0.05 were considered significant. Post hoc comparisons were made using the least significant difference test. Analyses within groups at different time points were done using t-test for dependent samples. Data from LF animals were not included in statistical analyses.

### Results

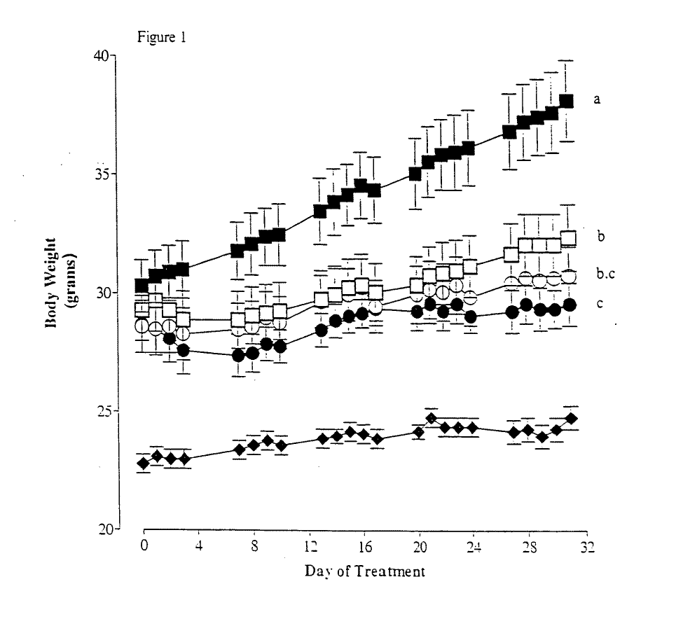

Body Weight. Food Intake, Feed Efficiency. The body weights of all groups consuming the HF diet were similar at the end of the pretreatment period. After administration of the compounds for 4 weeks, ANOVA showed a significant effect of treatment on body weight (p<0.001). As shown in FIG. 1. all three therapies reduced body weight gain when compared to the HF control group. At the end of the study, the combination therapy was more effective than the CL alone, but not more effective than Dz alone. Immediately following the change from HF to HF + drugs, there was a modest and brief (less than 4 days) decrease in food intake. However. ANOVA showed that total food intake tended to be influenced by treatment (p=0.08). Animals fed the diet containing CLDz ate more.

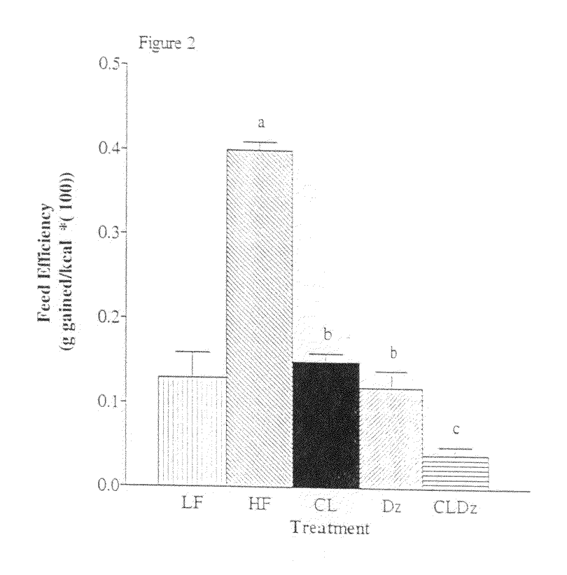

FIG. 2 illustrates that all treatments reduced feed efficiency when compared to HF (ANOVA p<0.001). Feed efficiency is the ratio of weight gained to calories consumed. It therefore reflects metabolic efficiency and is not confounded by caloric intake. CLDz was more effective than either CL or Dz in decreasing feed efficiency. Thus, even though the CLDz treated animals showed similar decreases in fasting insulin, they gained less weight.

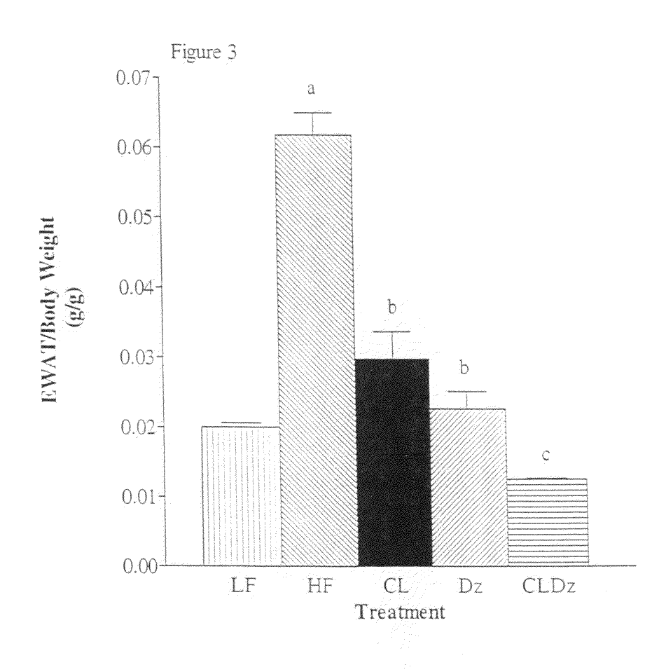

Estimate of Percent Body Fat. The effect of treatment on percent body fat is shown in FIG. 3. The weight of the EWAT fat pad as a proportion of total body weight was used to estimate the percent body fat. Percent body fat was significantly affected by treatment (ANOVA p<0.001). Importantly, CLDz was significantly more effective in decreasing percent body fat than either CL or Dz.

Fat Pad Weight. The wet weights of RP and EWAT fat pads are shown in Table 1. Both fat pads were significantly affected by treatment (ANOVA p<0.001 for both fat pads). RP fat pad weight was reduced by all treatments when compared to the HF group (p<0.001 for all treatments). CLDz was found to be more effective in decreasing RP weight than CL (p<0.01), but was as effective as Dz (p=0.51). Similarly. all treatments reduced the wet weight of EWAT fat pads when compared to the HF group (p<0.001 for all treatments). In addition, CLDz was significantly more effective than CL (p<0.01) and tended to be more effective (p=0.08) than Dz in reducing EWAT weight.

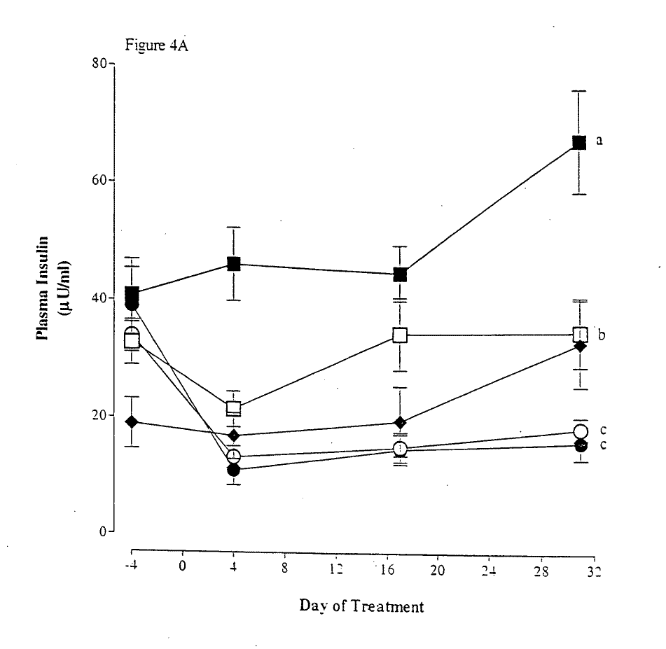

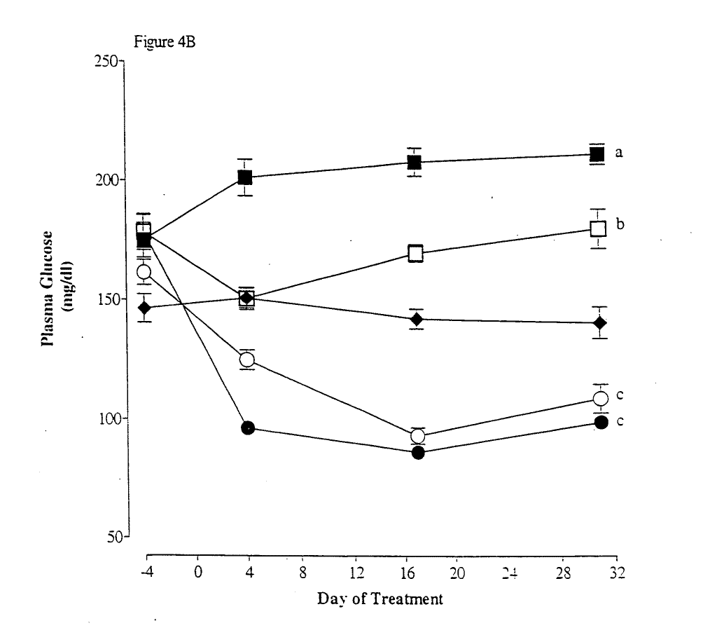

Insulin and Glucose. The effects of these various treatments on fasting plasma insulin and glucose over the course of the study are illustrated in FIG. 4. Within 4 days there was a significant effect of treatment on plasma insulin as shown by ANOVA (p<0.001). Insulin levels (FIG. 4A) were significantly decreased in all treated groups when compared to HF and when compared to their respective pretreatment values. At the conclusion of the study, ANOVA indicated a significant effect of treatment on plasma insulin (p<0.001). Insulin levels in the BF group continued to increase throughout the study; and they remained significantly higher when compared to any of the treatments (p<0.01 for all treatments). Initially the insulin levels within the CL group decreased; however, by the end of the study they were similar to the pretreatment value, even though they were lower than the HF group. By contrast, insulin levels in the Dz and CLDz groups remained significantly below pretreatment levels throughout the study. There appeared to be no added benefit of CLDz.

Within 4 days of instituting the drug regimens, there was a significant effect of treatment on plasma glucose (ANOVA p<0.001). FIG. 4B shows glucose levels in all treatment groups dropped significantly when compared to the HF control group. In addition, there was an immediate added benefit of CLDz in lowering plasma glucose levels. At day 4. glucose values in the CLDz group were significantly less than values for either treatment alone. The normalization in fasting plasma glucose values in the CL group was transient. Similar to the insulin levels in this group, plasma glucose had returned to the pretreatment values at the termination of the study. In contrast, fasting plasma glucose concentrations in the Dz and CLDz groups remained lower than pretreatment levels throughout the study.

Plasma Lipids and Leptin. Values for these parameters at the conclusion of the study are shown in Table 1. Treatment significantly affected plasma leptin (ANOVA p<0.001). Leptin levels were lower in all groups when compared to the HF control. Although CL decreased leptin levels when compared to the HF control, it was less effective for lowering leptin levels than either Dz or CLDz (p<001 for both comparisons).

Plasma triglycerides were also affected by treatment (p<0.001). While CL treatment significantly raised triglycerides when compared to the HF control group (p<0.05), Dz and CLDz lowered triglyceride levels (Dz p<0.01; CLDz p<0.001). In addition, all treatments were effective in lowering. NEFA in plasma when compared to HF-fed mice (CL p<0.001, Dz p<0.05, CLDz p<0.001), but CLDz was significantly more effective than either CL (p=0.01) or Dz (p<0.001).

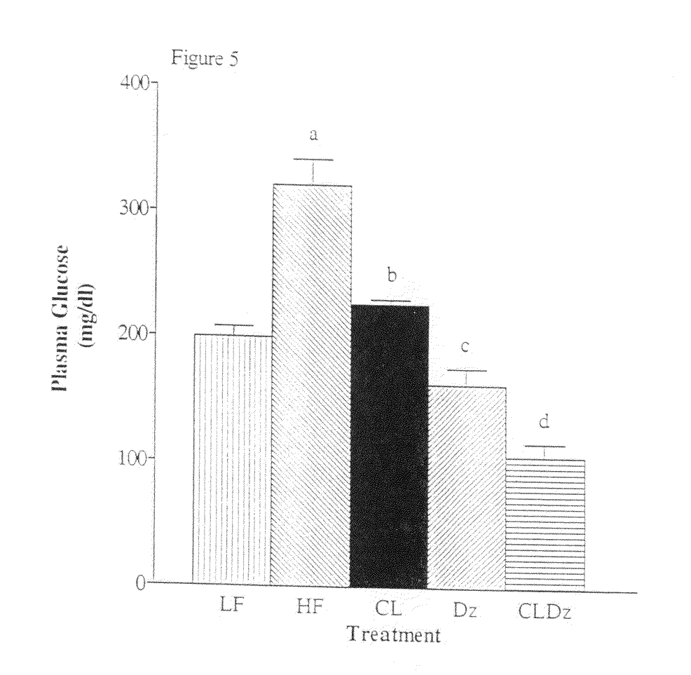

Glucose Tolerance. The mice were challenged with an i.p. bolus of glucose. FIG. 5 shows the results of treatment on glucose tolerance. CLDz was more effective than either CL or Dz in improving glucose tolerance. In animals treated with CLDz, plasma glucose was returned to the post-absorptive level within 30 minutes after the challenge (98 mg/dl post-absorptive vs. 106 mg/dl post challenge).

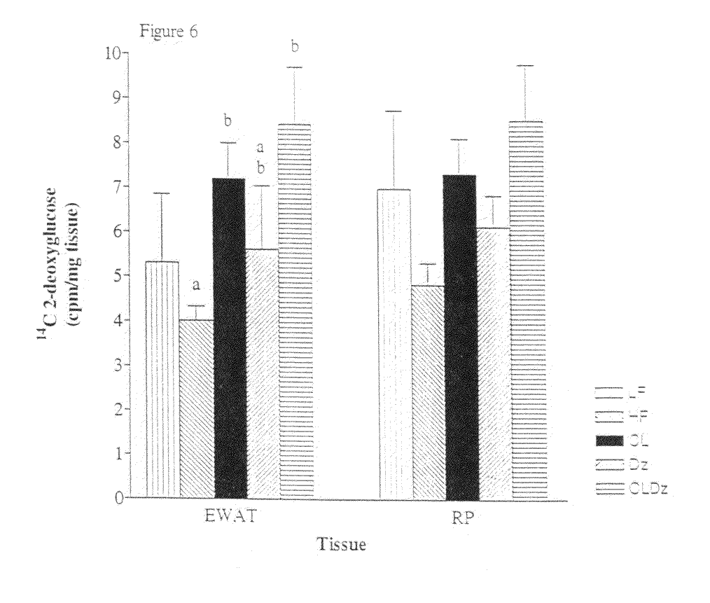

Glucose Transport into Adipose and Muscle Tissues. The efficacy of treatment on glucose transport into EWAT and RP fat pads is shown in FIG. 6. There was a significant effect of treatment on glucose transport into EWAT (ANOVA p<0.05). CL and CLDz improved glucose transport into EWAT when compared to the HF control group, but treatment with Dz did not reach a statistically significant level. With regard to the effect of these compounds on glucose transport into RP, ANOVA revealed a trend (p=0.07) toward a treatment effect. As seen in the figure, CL and CLDz enhanced glucose transport when compared to the HF control group. However, there was no apparent effect of treatment on glucose transport into muscle tissues.

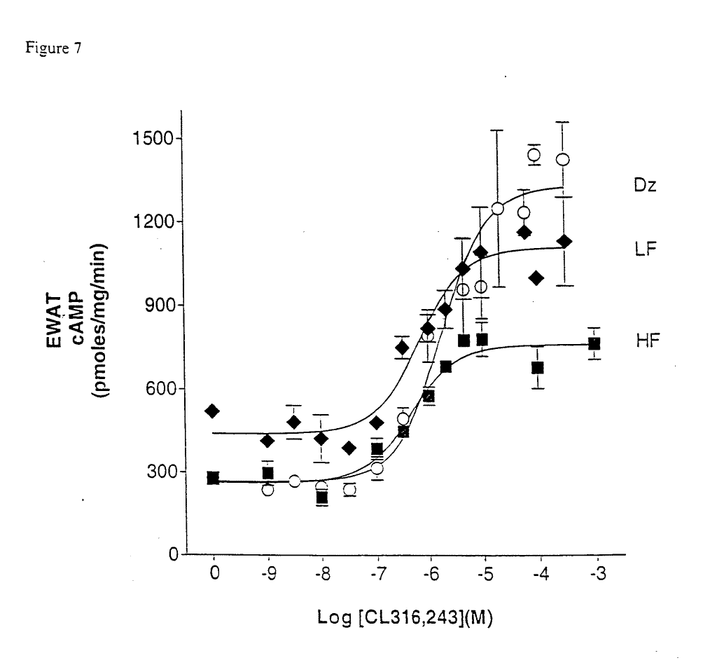

Adipocyte β₃AR function. It has been previously shown that β₃AR-stimulated adenylyl cyclase activity in EWAT is severely blunted in B6 mice on a HF diet (Collins et al, Endocrinology 138:405-413 (1997)). However, since the wet weight of the epididymal fat pads (and RP, not shown) from CL-, Dz-, and CLDz-treated animals were significantly less than those from their HF counterparts (FIG. 3), it suggested that lipolysis of previously stored lipids had occurred in each case. Therefore, plasma membranes from EWAT for were examined adenylyl cyclase activity in response to CL, since βAR-stimulated cAMP production is the major proximal step regulating lipolysis, (Robinson et al, In Cyclic AMP: Academic Press, New York (1971)). The means of three experiments in each condition are shown in FIG. 7. Non-linear regression analysis revealed that all three curves were significantly different from each other (p<0.0003). As previously observed in obese animals, (Collins et al, Endocrinology 138:405-413 (1997), Collins et al. Mol. Endocrinol. 8:518-527 (1994)) basal and β₃AR-stimulated adenylyl cyclase activity was significantly depressed in HF-fed B6 mice when compared to the LF animals (p<0.0001). Treatment with Dz not only reduced plasma levels of insulin and glucose, but also restored the efficacy of the β₃AR to stimulate adenylyl cyclase (p<0.0001). This is consistent with an increased ability to recruit stored lipids through this pathway. When similar measurements were made in adipocyte plasma membranes from CL and CLDz treated animals, a general decrease in adenylyl cyclase activity was encountered, which has been seen before in animals chronically treated with CL (Collins et al. Endocrinology 138:405-413 (1997)). The molecular basis of this phenomenon is still unclear but presumed to be some form of partial down-regulation of the signaling cascade.

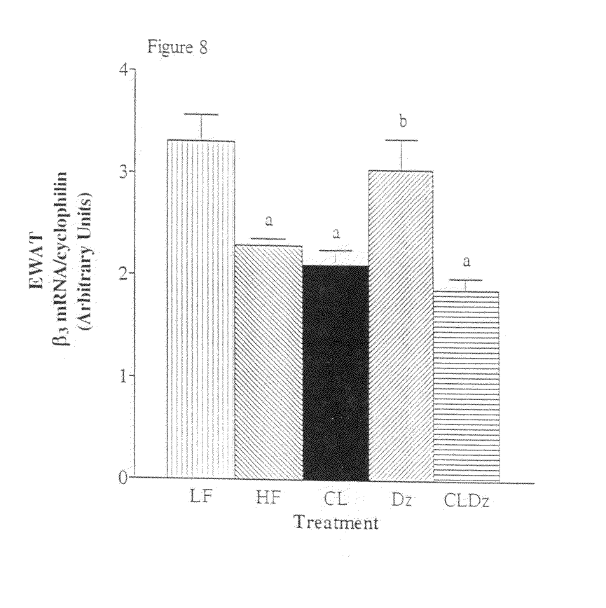

β₃AR expression in EWAT. Next it was determined whether the observed changes in β₃AR function in adipose tissue, as defined by adenylyl cyclase activity, were accompanied by concomitant alterations in β₃AR mRNA expression. The HF diet significantly reduced the expression of β₃AR mRNA in EWAT when compared to the LF control (p<0.01), as observed previously (Collins et al, Endocrinology 138:405-413 (1997)). FIG. 8 shows that animals treated with Dz alone expressed β₃AR mRNA at levels comparable to the LF fed controls. Similar to previous studies (Collins et al, Endocrinology 138:405-413 (19971), B6 mice treated with CL failed to restore β₃AR expression or function. FIG. 8 also shows that there was no improvement in β₃AR mRNA levels in CLDz treated animals. This result is confounding in view of the fact that WAT from these animals exhibited the greatest response to treatment in terms of decreased adipose tissue mass. However, in spite of the fact that lack of understanding of the apparent loss in mRNA expression of the β₃AR that is routinely observed in β₃AR-agonist treated animals, the physiological effect on total body weight and fat mass is clearly improved in these combination-treated animals.

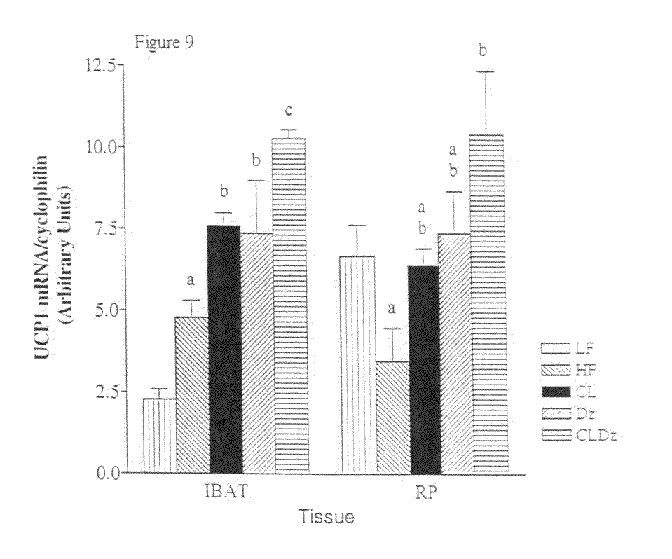

Regulation of brown adipocyte UCP1. The expression of UCP1 is specific to brown adipocytes and thus serves as a marker for the presence and thermogenic activity of IBAT (Bouillaud et al, J. Biol. Chem. 258:11583-11586 (1984)). Applicants (Collins et al, Endocrinology 138:405-413 (1997)) and others (Champigny et al, Proc. Natl. Acad. Sci. USA 88:10774-10777 (1991), Himms-Hagen et al, Diabetes 3:59-65 (1996)) previously showed that the ability of β₃AR agonists to prevent or reverse obesity is linked to the expansion of brown adipocytes and UCP1 expression, particularly in WAT depots, and varies between strains of mice (Collins et al, Endocrinology 138:405-413 (1997)). Therefore, examination was made of how the expression of UCP1 was affected in various adipose depots in B6 mice in response to a HF diet and subsequent treatment with Dz, CL, and CLDz. As shown in FIG. 9 and consistent with previous studies (Collins et al, Endocrinology 138:405-413 (1997), Surwit et al, Proc. Natl. Acad. Sci. USA 95:4061-4065 (1998)), levels of UCP1 mRNA were increased in IBAT in response to the HF diet compared to the LF control group (p<0.01). Animals treated with either CL or Dz exhibited a greater increase in UCP1 expression (p<0.05, p<0.01), respectively, while animals treated with the combination therapy showed a markedly greater elevation in UCP1 mRNA levels (p<0.001) when compared to the HF control. In the RP depot, which consists of a mixture of white and brown adipocytes (Cousin et al, J. Cell Sci. 103:931-942 (1992)), there was a modest decrease in UCP1 expression in response to HF feeding when compared to the LF control (p>0.05), consistent with previous findings (Collins et al, Endocrinology 138:405-413 (1997)). In HF animals receiving CL. Dz, or CLDz, only those treated with the combination therapy showed a significant increase in UCP1 expression (p<0.05). Only HF-fed B6 mice treated with Dz showed a significant increase in UCP3 expression in IBAT (p<0.05), while there was no effect of treatment on UCP2 expression in IBAT. It should be noted that increases in mRNA usually, but do not necessarily, indicate corresponding changes in protein levels. Measurement of protein levels of β₃AR and UCP1 was not possible.

All documents cited above are hereby incorporated in their entirety by reference.

One skilled in the art will appreciate from a reading of this disclosure that various changes inform and detail can be made without departing from the true scope of the invention.

TABLE 1

| | | | | Epididymal | Retroperitoneal |
|---|---|---|---|---|---|
| | Leptin | NEFA | Triglycerides | Fat Pad Weight | Fat Pad Weight |
| | (ng/ml) | (mEq/L) | (mg/dl) | (grams) | (grams) |
| LF | 5.5 +/− 1.8 | 0.90 +/− 0.05 | 81 +/− 4 | 0.505 +/− 0.018 | 0.108 +/− 0.006 |
| HF | 27.1 +/− 2.0^{a} | 0.99 +/− 0.04^{a} | 126 +/− 7^{a} | 2.340 +/− 0.177^{a} | 0.414 +/− 0.058^{a} |
| CL | 15.7 +/− 2.1^{b} | 0.69 +/− 0.04^{b} | 148 +/− 8^{b} | 0.943 +/− 0.129^{b} | 0.200 +/− 0.024^{b} |
| Dz | 4.2 +/− 0.8^{c} | 0.82 +/− 0.08^{b} | 96 +/− 7^{c} | 0.691 +/− 0.083^{b,c} | 0.123 +/− 0.014^{b,c} |
| CLDz | 4.8 +/− 1.1^{c} | 0.51 +/− 0.03^{c} | 82 +/− 5^{c} | 0.377 +/− 0.005^{c} | 0.092 +/− 0.001^{c} |

Treatments with different superscripts are significantly different.
Leptin: a vs. b or c, p < .001; b vs. c, p < .001
NEFA: a vs. b, p < .05; a vs. c, p < .001; b vs. c, p < .05
Triglycerides: a vs. b, p < .05; a vs. c, p < .01; b vs. c, p < .001
Epididymal Fat Pad: a vs. b or c, p < .001; b vs. c, p < .01
Retroperitoneal Fat Pad: a vs. b or c, p < .001; b vs. c, p < .05

What is claimed is:

1. A method of increasing the sensitivity of a hyperinsulinemic patient to the anti-obesity and antidiabetic effects of a β adrenergic receptor (βAR) agonist comprising administering to said patient said βAR agonist and an agent that reduces plasma insulin levels under conditions such that said increase is effected.

2. The method according to claim 1 wherein said βAR agonist is CL316,243.

3. The method according to claim 1 wherein said agent is diazoxide.
